(19) United States
(12) Patent Application Publication
(10) Pub. No.: US 2012/0234089 A1
(43) Pub. Date: Sep. 20, 2012

(54) BUOYAGE AND ENVIRONMENTAL MONITORING INSTRUMENT FOR USE ON REGATTA COURSES

(76) Inventors: Octavio Llinas Gonzalez, Las Palmas (ES); Maria Jose, Las Palmas (ES); Carlos Barrera Rodriguez, Las Palmas (ES); Maria Guadalupe Villagarcia Ubeda, Las Palmas (ES); Laura Cardona Diaz, Las Palmas (ES)

(21) Appl. No.: 13/459,752

(22) Filed: Apr. 30, 2012

Related U.S. Application Data
(63) Continuation-in-part of application No. 11/814,948, filed on Sep. 9, 2009.

(30) Foreign Application Priority Data
Jan. 28, 2005 (ES) P200500293

(51) Int. Cl. B63B 35/44; G01W 1/00; B63B 22/24; G01N 33/18; B63B 22/16; B63B 22/22

(52) U.S. Cl. 73/170.16; 114/264; 73/170.29

(57) ABSTRACT

The present invention relates to an instrument for marking and environmental monitoring in regatta courses having an inflatable float and a modular mast which is collapsible, The mast and float are transportable in compact package of reduced size. When deployed, the instrument is designed to simultaneously define the area of conducting the nautical trials, and transmit the conditions of the area in both meteorological and oceanographic terms. The system comprises an inflatable pneumatic float and a mast which serves to house the meteorological and oceanographic instrumentation, as well as a group of elements for communication, electromechanical components, and an anchorage system.

### RELATED APPLICATIONS

The present application is a continuation-in-part of U.S. patent application Ser. No. 11/814,948, which was a national phase application of PCT No. PCT/ES2006.000038 filed Sep. 9, 2009, claiming priority of Spanish Application No. P200500293 filed Jan. 28, 2005.

### FIELD OF THE INVENTION

The present invention is framed in a parallel fashion within two technical sectors of the marine environment: on one hand, the marine buoys or markers employed in regatta courses of competitive sailing, and on the other, the monitoring of environmental parameters in real time with direct application, in this case, and without intending to be restrictive in the scope thereof, to the sport of competitive sailing.

### STATE OF THE ART

The advances in technology that our society experiences on a daily basis, are reflected particularly in the sport sector. This occurrence means that the practice of most sports is benefitting from the multidisciplinary nature of technology found on the market at the present time. Within this section, competitive sailing is in no way an exception.

All the disciplines in competitive sailing are undertaken in a similar geographical framework, under determined environmental conditions. That is, all have the need of a buoyed regatta course, the dimensions of which have to be in agreement with the discipline or modality pursued and, also, there is a need to know in the fullest measure possible the environmental parameters that condition these sporting events (mainly, wind direction and strength and surface current of the sea).

The delimitation or marking of these regatta courses, has to be carried out with floating platforms or buoys, of certain constructional characteristics (material, color, dimensions, location regarding the bottom, etc.), although there is no specific legislation existing at present to such effect. It is only necessary to take into account the generic legislation as regards anchorage restrictions in certain areas (the environs of harbor areas, areas with heavy marine traffic, naval maneuvers, marine reserves, etc.), generic regulations on marine signaling as well as the particular opinion of the marine master of the area in question.

To date, the document considered pertinent is U.S. Pat. No. 3,828,380 which discloses a floating instrument for the study of oceanic environmental conditions. It has an elongated spar or mast (like the Instrument of the present invention), but said mast is thicker and heavier in the bottom part, besides having a platform on the upper part where the antenna is located. In our case, the mast is also comprised of several modules and is collapsible, but unlike the former, it maintains the same diameter over all its length. The most important difference between the two lies in the means of flotation for the mast. In the document U.S. Pat. No. 3,828,380 the means of flotation is annular and is secured to the mast by means of some flexible wires and is positioned over the center of gravity of the same. This signifies that the mast has freedom of movement within some limits, moving above and below the means of flotation. In our case, the float is rigidly mounted on the mast, reducing the movement of the antenna due to currents and waves, and thereby avoiding in a greater measure the possibility of the mast being dunked. The operation of the patented device is much more complicated, because it needs a pressurized gas for the inflation of the flotation element and for the deployment of the mast. On the other hand, in the present invention an energy source can be used from an ecological or alternative source for charging or recharging the batteries and/or accumulators.

It is also worthwhile to mention the document U.S. Pat. No. 5,132,699 which discloses a Instrument for transmitting the oceanic environmental conditions by radiofrequency. The structure in this patent differs in form from the mast of the present application, because although it is true that both have the means of communication therein, the patented instrument is composed of two antenna structures which are angular in shape. This structure is made of an electrical conductor.

The present invention seeks to integrate the marking and delimitation of the course with the continuous monitoring in real time of the environmental parameters of interest and conditions for the optimum development of competitions in anyone of the presently existing sailing classes.

### SUMMARY OF THE INVENTION

The present invention relates to an instrument incorporated by an inflatable pneumatic platform and modular mast, collapsible and transportable in a compact package of reduced size and easy to handle, intended for the marking and monitoring of environmental parameters in real time of regatta courses in competitive sailing. It is sought simultaneously to define the area of undertaking such nautical trials, at the same time as their environmental conditions are characterized, in real time and in continuous mode, in meteorological and oceanographic terms.

Attention is specially drawn to the highly modular character and the reduced weight of the Instrument as a whole, which simplifies the transportation thereof using a compact packing system (for example, suitcase type of reduced size.

This simplicity in its handling extends to the mounting and emplacement of the instrument in the marine environment, it being possible in this case, to carry out quickly a modification in the delimitation of the regatta course.

This aspect is of great importance, due to the high degree of variability that these areas suffer during the undertaking of the trials as a consequence of a sudden variation in environmental conditions (mainly those referring to the wind.

### DESCRIPTION OF THE FIGURES

FIG. 1 is a diagrammatic view of the invention in side elevation with the right side portions broken away to illustrate a longitudinal section of the interior of the right side;

FIG. 2 is a diagrammatic view of the instrument in elevation, showing protective elements on both ends of the mast during the transport of the Instrument; and

FIG. 3 is a diagrammatic view of the instrument when the mast is collapsed and the flotation element is deflated and compressed into a compact package of reduced size and inserted in a carrier.

### DESCRIPTION OF A PREFERRED EMBODIMENT

The Instrument comprises two differentiated units, namely, an inflatable pneumatic float 1 and a support mast 2, the latter serving to hold the meteorological instrumentation 3 and oceanographic instrumentation 6, as well as the group of elements intended for communications 4, electromechanical components 5, and a system 7 for securing the instrument to an anchoring train (not illustrated).

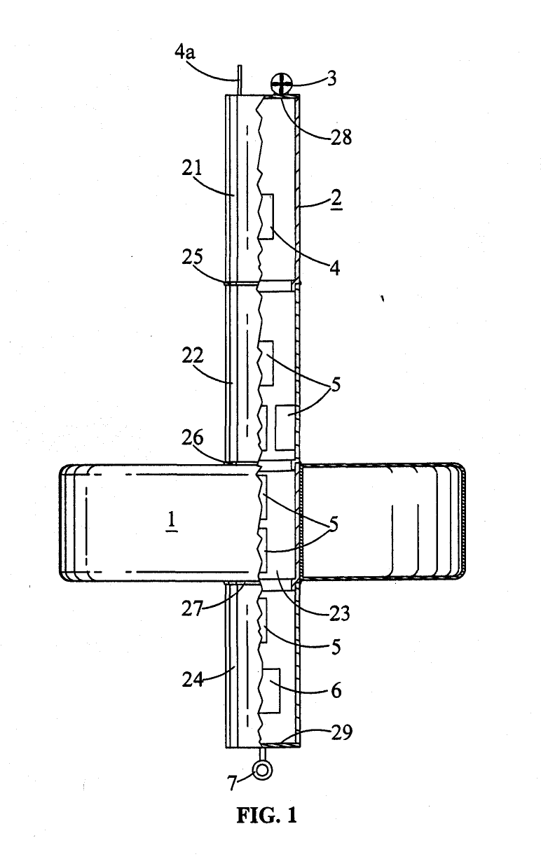

The float 1, is a structure composed of a pneumatic or plastic material forming a donut-shaped bladder shown in FIG. 1, which has the particular characteristic of being inflatable or deflatable as required. Its basic functions are (a) to give the necessary buoyancy to the Instrument as a whole and moreover, and (b) to allow the extremities that define the designated regatta course area to be viewed. This float 1, the physical characteristics of which are defined by the standards that regulate the type of sport activity in question size, heat, etc., is located approximately in the center of the longitudinal plane of the Instrument as a whole. The reason for this location responds to the needs imposed by the Instrument regarding equilibrium and buoyancy.

The float 1 is crossed by the hollow support mast 2 which is perpendicular to the surface of the body of water providing the regatta course, and serves to hold the meteorological instrumentation 3 and oceanographic instrumentation 6 as well as the group of elements intended for communications 4 and other electromechanical components 5.

The dimensions of the supporting mast 2 may be varied, depending upon the type of measurement instrumentation to be mounted therein or thereon. It is constituted by several modules 21, 22, 23 and 24, each one of which is intended to hold a certain element or electronic component, thereby offering a higher degree of modularity and simplicity for both the assembly and the transportation. The mast is collapsible by reason of the presence of separable connections 25, 26, and 27 connecting the modules. The separable connections may be hinges or telescoping joints. As for the arrangement of modules on this mast, the upper exposed extremity of the upper module 21, has a platform 28 mounting the exposed meteorological instrumentation 3 and a transmitting antenna 4a, the communications module 4 is mounted within the module below the platform 28. In the illustrated embodiment, the meteorological instrumentation 5 is mounted within the modules 22 and 23. The oceanographic instrumentation 6 is located either in the lower submerged module 24, as shown, or may be mounted in the lower part of the float 1.

The operation both of the meteorological instrumentation 3 and the oceanographic instrumentation 6 may be varied, although in whatever case, the instrumentation responds at all times to the international standards proposed by the competent organizations, as regards environmental monitoring.

The communications system 4 is housed inside of the hollow support module 21, with the exception of the antenna element, which shares the platform 28 with the meteorological instrumentation 3. The communication system between the measurement platform and land is implemented, preferably via satellite, by the electromechanical components 5, which include a radiofrequency transmitter device and/or a satellite-signal transmitting device.

The electromechanical components 5 also include a power supply source that supplies the communication system 4 and the instrumentations 3 and 4 of the Instrument. The power supply may be constituted by batteries and/or accumulators, which have the additional option of being charged and/or recharged by an alternative and ecological energy sources, such as photovoltaic cells and/or wind turbines.

All the devices or elements that comprise the Instrument , have a level of watertightness corresponding to IP68, which guarantees protection in greater measure from the adverse environmental conditions habitually encountered in maritime surroundings.

Given the modular character which the Instrument offers, it is possible to include other devices in the event they are necessary, such as night- and day-signaling elements, complying in this way with the regulations in force regarding marine signaling.

In FIG. 1 it is possible to observe the entirety of the elements that form the Instrument, namely, the float 1, which is crossed lengthwise in the vertical plane, by the supporting mast 2 which serves to hold the meteorological Instrumentation 3 and oceanographic Instrumentation 6, as well as the group of elements dedicated to communications 4 and the other electromechanical components 5.

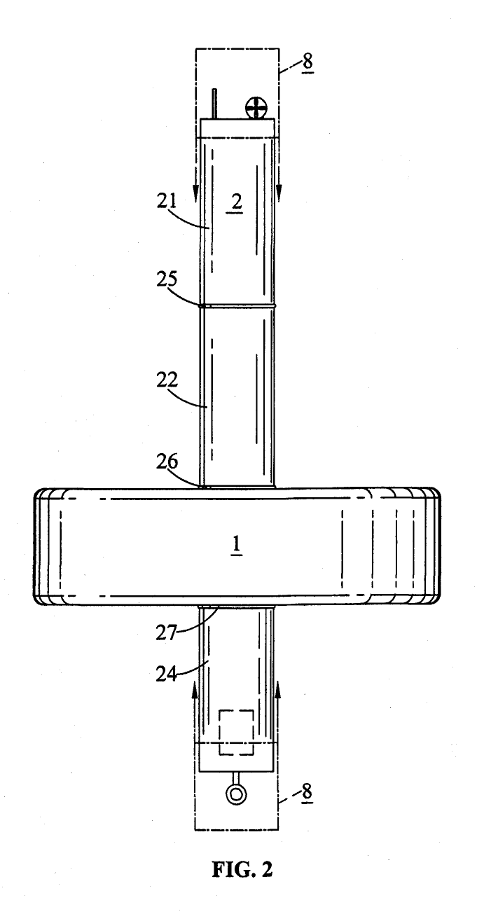

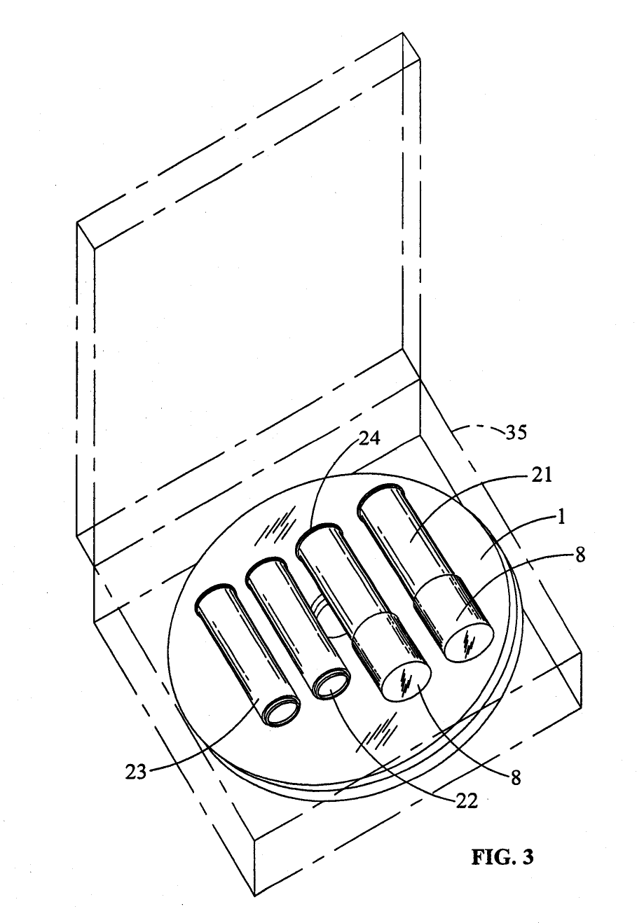

FIG. 1 shows the segmentation or modularity of the mast 2 and the rigid mounting of the float 1 on the module 23. The uppermost and lowermost modules 21 and 24 of the mast 2 have an accessory element 8 (see FIGS. 2 and 3) for protection during assembly, disassembly and transport, which increases the safety of the meteorological 3 and oceanographic 4 Instrumentation during the transportation of the instrument as a whole by means of a carrier 35 (see FIG. 3).

In FIG. 1 it is possible to observe the presence of a securing element 7, located on a platform 29 at the lower end of the mast 2, intended for securing the instrument to the pertinent anchor train (not illustrated).

### Operation of Embodiment of the Invention

The present invention is explained by means of the following embodiment, although this is not intended to restrict the scope thereof.

In the light of the elements already described that form the present invention, a method of assembling and operating the same as a whole could be described in the following manner:

In the first place, it is necessary to consider the support structure mast divided into several modules which may be varied in number, although four modules are in the illustrated embodiment. The high module 21 mounts the meteorological instrumentation 3 and the communications antenna 4a of the communication system 4. The electromechanical components 5 are mounted within the second, third and fourth modules 22, 23 and 24. The third module 23 mounts the float 1. The lower fourth module 24 contains the oceanographic instrumentation 6 as well as selected electromechanical components 5, all of the modules mating with each other in a certain sequence. The modules of the mast 2 are interconnected by separable connections 25, 26 and 27 to enable the mast to be collapsed into smaller dimensions for storage or transport. As illustrated in FIG. 3, the float 1 is deflated and flattened to fit into a carrier, shown in broken lines 35. It should be noted that the carrier 35 has an internal width greater than the overall width of the float and an internal height greater than the smaller height of the collapsed float plus the thickness of the mast modules. To facilitate packing the instrument, all of the modules 21,22, 23, and 24 have length less than the overall width of the float 1. In the illustrated embodiment, the mast modules 21, 22 and 24 are of the same length, but module 23 is shorter than the others.

When unpacking the carrier 35 and assembling the Instrument, the starting point taken will be the third module 23, on which the assembly of the remaining modules will be carried out, so that the incorporation of these is as simple as possible for the user. For this reason, the following module to assemble has to be the lower module 23. Having connected the oceanographic instrumentation 6 to the central processing device of the electric mechanical components 5 and closed the connection 27 between the two modules. The assembling continues with the upper module 21, having to be connected to the module 22 through the upper end of the latter. With this, it is managed to connect the group of meteorological sensors 3 and communication devices 4, 4a to the flotation module, where habitually the electronic devices 5 are housed intended for the management and optimum operation of the Instrument as a whole. The interconnected modules 21 and 22 are then connected to the upper end of the module 23, to leave the Instrument completely assembled.

Having assembled the four modules as described, the Instrument will be conveyed to the definitive anchorage. At this point, the inflation of the float will take place, in the event of this not having been performed before embarking in the vessel employed for its transfer to the selected anchorage point. In both cases, an external source of pressurized air will be required for inflating the float.

Finally, the instrument will be secured to the selected anchorage train by means of a fastening system 7 located on a platform 29 at the lower end of the last module 24 of mast 2.

### INDUSTRIAL APPLICATION

The present invention relates to an Instrument with application both in the sport of sailing and in the monitoring in real time of environmental parameters in the marine environment.

To such an end, a number of examples of industrial applications have been set forth, which do not intend to be restrictive in the scope thereof.

Thus, the relevance is specified that the invention signifies in the sporting sector of competitive sailing, in the full gamut of categories therein, allowing the continuous knowledge of the conditions in real time that regulate the regatta courses, at the same time as it defines the area designated for the holding of such events.

In parallel, the competent institutions and organizations in this matter, could take the present invention into consideration, as an indispensable element by way of Infrastructure necessary for carrying out the holding of these sporting events in optimum conditions. Thus, we understand that given the number of institutions and organizations in this environment, as well as the continuous upsurge that the sport of sailing is experiencing worldwide, the present invention offers an important line of industrial application.

1. Instrument for marking a regatta course in a body of water and measuring environmental parameters, said instrument adapted to float at the surface of the body of water and comprising a collapsible mast comprising a plurality of hollow modules operable between a collapsed condition and a deployed condition, said mast, when deployed adapted to be disposed perpendicular to the surface, said instrument modules mounting a communications device with a transmitter of signals, a parameter-measuring device, and an inflatable and deflatable pneumatic-plastic float, said float, when deflated, being both collapsible to smaller dimensions, and transportable in compact carrier of reduced size,
characterized in that when said mast is deployed perpendicular to the surface, said mast has the same thickness over all the deployed length thereof, and
said pneumatic float is rigidly attached to the mast approximately in the center of the length thereof.

2. The instrument of claim 1 wherein said communications device comprises a radiofrequency transmitter.

3. The instrument of claim 1 wherein said communications device is operative to generate signals to communicate via satellite.

4. The instrument of claim 1, characterized in that said parameter measurement devices comprise meteorological and oceanographic instrumentation, and electromechanical components including a stand-alone power supply system.

5. The instrument of claim 4 wherein said communication devices include electromechanical components.

6. The instrument of claim 1, characterized in that it incorporates a mast support structure with a power supply system which is alternative and ecological in nature.

7. The instrument of claim 6 wherein the power supply comprises photovoltaic cells.

8. The instrument of claim 1, characterized in that said mast has upper and lower extremities, and at least one of said extremities has a support adapted to mount an easily mounted protective system for said instrumentation, said support and said protective system being adapted to permit said instrumentation to be usable during the transport thereof.

9. The instrument of claim 1, characterized in that it is adapted to mark an area of conducting nautical trials, said instrumentation and said communication device being operable to transmit the parameters measured by said parameter-measuring device in real time and in continuous mode, in both meteorological and oceanographic terms.

10. Instrument for marking a regatta course in a body of water and measuring environmental parameters, said instrument adapted to float at the surface of the body of water, said instrument comprising
a collapsible mast comprising a plurality of modules operable between a collapsed condition and a deployed condition, said mast, when deployed adapted to be disposed perpendicular to the surface, said instrument modules having hollow interiors mounting a communications device with a transmitter of signals, and parameter-measuring devices,
an inflatable and deflatable pneumatic-plastic float having an overall width and a given height mounted on the exterior of said instrument modules, said pneumatic float being is rigidly mounted on the mast approximately in the center of the length thereof, said float, when deflated and dismounted from said modules, is both collapsible to smaller height and transportable in compact carrier of reduced size,
when said mast is deployed, said float supports said mast perpendicular to the surface, and said mast has the same thickness over all the deployed length thereof, and
when said mast is collapsed, each of said modules has a length less than the overall width of said float.

11. The Instrument of claim 10 wherein all of said mast modules have the same uniform thickness, in combination with a carrier having an internal width greater than said overall width of the float and having an internal height greater than said smaller height of the collapsed float plus said uniform thickness of the mast modules.
